(19) United States
(12) Patent Application Publication
(10) Pub. No.: US 2014/0180068 A1
(43) Pub. Date: Jun. 26, 2014

(54) CATHETER ORIENTING MARKERS

(71) Applicant: VOLCANO CORPORATION, SAN DIEGO, IL (US)

(72) Inventors: Jason Spencer, Rocklin, CA (US); Russell W. Bowden, Tyngsboro, MA (US); Stan Thomas, San Diego, CA (US); Oren Levy, Emerald Hills, CA (US); Byong-Ho Park, San Diego, CA (US); Dietrich Ho, San Diego, CA (US)

(73) Assignee: VOLCANO CORPORATION, SAN DIEGO, CA (US)

(21) Appl. No.: 14/134,539

(22) Filed: Dec. 19, 2013

Related U.S. Application Data
(60) Provisional application No. 61/740,762, filed on Dec. 21, 2012.

(51) Int. Cl. A61B 5/06; A61B 8/08; A61B 5/00

(52) U.S. Cl. CPC A61B 5/064; A61B 5/0073; A61B 8/0841
USPC 600/424; 600/431

(57) ABSTRACT

The present invention generally relates to methods, devices and systems for determining the rotational orientation of a device. The invention can involve providing a device comprising a plurality of markers, wherein each marker within the plurality of markers differs from an adjacent marker by size, shape, and/or position on the device. The invention can also involve inserting the device into a vessel and imaging the device to capture an image of the device in an imaging plane. The invention can further involve processing the captured image to determine an orientation of the device relative to the imaging plane based on the markers.

### RELATED APPLICATION

This application claims the benefit of and priority to U.S. Provisional Ser. No. 61/740,762, filed Dec. 21, 2012, which is incorporated by reference in its entirety.

### FIELD OF THE INVENTION

The present invention generally relates to systems, methods, and devices for determining the rotational orientation of an imaging catheter.

### BACKGROUND

Cardiovascular disease frequently arises from the accumulation of atheromatous deposits on inner walls of vascular lumen, particularly the arterial lumen of the coronary and other vasculature, resulting in a condition known as artherosclerosis. These deposits can have widely varying properties, with some deposits being relatively soft and others being fibrous and/or calcified. In the latter case, the deposits are frequently referred to as plaque. These deposits can restrict blood flow, resulting in myocardial infarction in more severe cases.

The assessment and treatment of cardiovascular disease often involves cardiac catheterization. In this medical procedure, a catheter is inserted into a chamber or blood vessel of the heart in order to diagnose or treat certain conditions. For example, the catheter may be used to image areas in which plaque has accumulated. In intravascular ultrasound (IVUS) imaging, an imaging catheter is threaded over a guidewire into a blood vessel and images of the surrounding areas are acquired using ultrasonic echoes. Subsequent treatment may involve angioplasty, stent delivery, or ablation.

When imaging with an internal imaging device such as an IVUS catheter, it is desirable in many instances to know the orientation of the obtained images. For example, when crossing a complete arterial blockage with an ablator, it is useful to know whether the ablation device is positioned up or down. As another example, knowing the orientation of an obtained image is also useful when imaging in areas where little is known about the structure, such as in a peripheral artery. Unfortunately, conventional methods and devices have yet to adequately address this need.

### SUMMARY

The present invention provides intraluminal devices comprising detectable markers that indicate the orientation of the device in the lumen. For example, the invention contemplates a catheter that contains a plurality of spaced-apart markers that determine the planar or rotational orientation of the catheter.

The markers may differ in size and shape; and they may be spaced apart at any convenient interval on the catheter. Devices, and associated methods, of the invention determine the rotational orientation of an intraluminal device in situ; and also determine the rotational orientation of an image captured by the device. For example, the invention is useful in determining the rotational orientation of an IVUS catheter as well as images obtained by the catheter using an external imaging modality, such as an angiogram. Accordingly, the invention significantly facilitates the diagnosis and treatment of cardiovascular disease where it is critical to know, for example, the orientation of a vessel imaged by a catheter or the orientation of an interventional device delivered over the catheter.

Any detectable marker may be used in connection with the invention. However, in a preferred embodiment, radiopaque markers are used. Once the orientation of the catheter has been determined, for example, by evaluating marker images on an angiogram, the proper orientation of the image obtained by the catheter is determined. Although the invention is suited for IVUS and IVUS catheters, the invention is equally applicable to other internal imaging modalities, such as optical coherence tomography (OCT). In addition, external imaging technologies amenable with the invention extend beyond angiogram fluoroscopy and can include, for instance magnetic resonance imaging (MRI). Forward imaging modalities are encompassed by the invention as well.

In one aspect, the invention encompasses an imaging catheter with a plurality of radiopaque markers that facilitate determining the orientation of the catheter and any images obtained by the catheter. More specifically, the configuration of the markers on the catheter, including their number, shape, size, and position on the device allows the orientation of the device to be determined. In certain aspects, the markers are offset from one another, which facilitates determining their orientation. The number, shape, size, amount of offset, and position can be adjusted as desired. In addition, the markers can be located on a component that is then attached to the catheter, rather than on the catheter itself.

In another aspect, the invention encompasses a method for determining the rotational orientation of an imaging catheter. The method can involve providing an imaging catheter with plurality of radiopaque markers configured in a manner that facilitates ascertaining the orientation of the device when the catheter is imaged externally. The method can further involve imaging the catheter externally to capture an image of the catheter in an imaging plane. The method also involves processing the captured image to determine the orientation of the catheter relative to the imaging plane. Additional aspects of the provided method involve orienting the image captured by imaging catheter based upon the previous orientation step.

In yet another aspect, the invention encompasses a system for determining the rotational orientation of an imaging catheter. The system can involve a processor and a computer readable storage medium having instructions that when executed, cause the processor to execute the methods of the invention. For example, the instructions may cause the processor to receive a captured image of an externally imaged catheter and process the captured image to determine the orientation of the catheter relative to the imaging plane of the captured image. The catheter, as described above, has a plurality of radiopaque markers configured in a manner that facilitates ascertaining the orientation of the device when the catheter is imaged externally.

### BRIEF DESCRIPTION OF THE DRAWINGS

FIG. 1 illustrates an exemplary imaging catheter for use in practicing methods of the invention.

FIG. 2A depicts a region featuring a plurality of markers on the exemplary imaging catheter depicted in FIG. 1.

FIG. 2B provides a forward-looking cross-sectional perspective of each marker within the plurality.

FIGS. 3A and 3B illustrate views of a single marker from an external imaging modality when the device is perpendicular to the imaging plane and parallel to the imaging plane, respectively.

FIGS. 4A and 4B depict an ultrasound image taken by an exemplary imaging catheter before and after using methods of the invention to determine the correct rotational orientation.

FIGS. 5A and 5B depict an exemplary imaging catheter and an enlarged view of an orienting marker configuration on the catheter, respectively.

FIGS. 5C and 5D depict the exemplary imaging catheter of FIGS. 5A and 5B in different rotational orientations.

FIGS. 6A and 6B depict another exemplary imaging catheter and an enlarged view of an orienting marker configuration on the catheter, respectively.

FIGS. 6C and 6D depict the exemplary imaging catheter of FIGS. 6A and 6B in different rotational orientations.

FIG. 7 depicts yet another exemplary imaging catheter.

FIG. 8 is a block diagram of an exemplary system for determining the rotational orientation of an imaging device.

FIG. 9 is a block diagram of an exemplary networked system for determining the rotational orientation of an imaging device.

### DETAILED DESCRIPTION

The present invention generally relates to devices, systems, and methods for determining the rotational orientation of a device that exploit particular arrangements of markers located on the device to thereby determine rotational orientation. The provided invention significantly facilitates the diagnosis and treatment of cardiovascular disease where it is critical to know, for example, the orientation of a vessel imaged by the catheter or the orientation of an interventional device delivered over the catheter.

Although the present invention can be practiced with any elongated body, in certain embodiments, the invention encompasses an imaging catheter or guidewire. Imaging may comprise any imaging modality, including, but not limited to intravascular ultrasound, intravascular Doppler, and intravascular optical coherence tomography (OCT). Moreover, any target can be imaged by systems and methods of the invention including, for example, bodily tissue. In certain embodiments, systems and methods of the invention image within the lumen of a tissue. Various lumen of biological systems may be imaged, including, but not limited to, blood vessels, vasculature of lymphatic and nervous systems, various structures of the gastrointestinal tract including the lumen of the small intestine, large intestine, stomach, esophagus, colon, pancreatic duct, bile duct, hepatic duct, lumen of the reproductive tract including the vas deferens, uterus and fallopian tubes, structures of the urinary tract including urinary collecting ducts, renal tubules, ureter, and bladder, and structures of the head and neck and pulmonary system including sinuses, parotid, trachea, bronchi, and lungs. The dimensions and other physical characteristics of the catheter or guidewire may vary depending on the body lumen that is to be accessed. In addition, the dimensions can depend on the placement and number of imaging elements included on the imaging catheter or guidewire.

When imaging vasculature, the imaging catheters are delivered to the tissue of interest via an introducer sheath placed in the radial, brachial, or femoral artery. The introducer is inserted into the artery with a large needle, and after the needle is removed, the introducer provides access for guidewires, catheters, and other endovascular tools. An experienced cardiologist can perform a variety of procedures through the introducer by inserting tools such as balloon catheters, stents, or cauterization instruments. When the procedure is complete, the introducer is removed, and the wound can be secured with suture tape.

The provided catheters and guidewires may also serve other functions in addition to imaging. In certain aspects, the provided catheter may also serve as a delivery catheter for delivery of some type of a therapeutic device, such as a stent, ablator, or balloon. During the procedure, the catheter may be used to identify the appropriate location and the delivery catheter used to deliver the device to the appropriate location. In certain embodiments, the provided guidewire may serve as rail for the introduction of a catheter. The catheter is slid over the provided guidewire and used as normal.

The guidewire used in accordance with the invention may include a solid metal or polymer core. Suitable polymers include polyvinylchloride, polyurethanes, polyesters, polytetrafluoroethylenes (PTFE), silicone rubbers, natural rubbers, and the like. Preferably, at least a portion of the metal or polymer core and other elements that form the imaging guidewire body are flexible.

Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques. Preferably, at least a portion of the catheter body is flexible.

In certain embodiments, the invention encompasses imaging tissue using intravascular ultrasound (IVUS). IVUS uses a catheter with an ultrasound probe attached at the distal end. The proximal end of the catheter is attached to computerized ultrasound equipment. To visualize a vessel via IVUS, angiography is used while the operator positions the tip of the guidewire. The operator steers the guidewire from outside the body, through angiography catheters and into the blood vessel branch to be imaged.

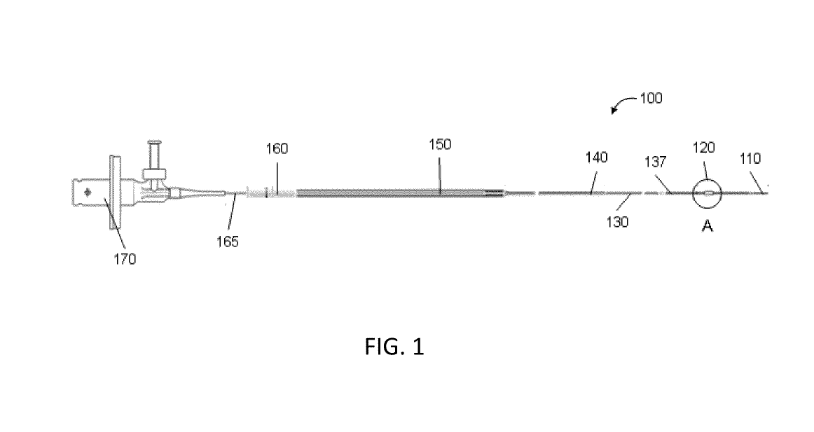

An exemplary IVUS catheter is shown in FIG. 1. Rotational imaging catetheter 100 is typically around 150 cm in total length and can be used to image a variety of vacualture, including coronary or carotid arteries and veins. When the rotational imaging catheter 100 is used, it is inserted into an artery along the guidewire (not shown) to the desired location. Typically a portion of the catheter, including the distal tip 110, comprises a lumen (not shown) that mates with the guidewire, allowing the catheter to be deployed by pushing it along the guidewire to its destination.

An imaging assembly 120 proximal to the distal tip 110, includes transducers 122 that image the tissue with ultrasound energy (e.g., 20-50 MHz range) and image collectors 124 that collect the returned energy (echo) to create an intravascular image.

Rotational imaging catheter 100 additionally includes a hypotube 140 connecting the imaging window 130 and the imaging assembly 120 to the ex-corporal portions of the catheter. Located distal to the imaging window is a plurality of radiopaque markers 137, discussed in more detail below. The hypotube 140 combines longitudinal stiffness with axial flexibility, thereby allowing a user to easily feed the catheter 100 along a guidewire and around tortuous curves and branching within the vasculature. The ex-corporal portion of the hypotube 140 can include shaft markers hat indicate the maximum insertion lengths for the brachial or femoral arteries. The ex-corporal portion of catheter 100 also include a transition shaft 150 coupled to a coupling 160 that defines the external telescope section 165. The external telescope section 165 corresponds to the pullback travel, which is on the order of 130 mm. The end of the telescope section is defined by the connector 170 which allows the catheter 100 to be interfaced to a patient interface module (PIM) which includes electrical connections to supply the power to the transducer and to receive images from the image collector. The connector 170 also includes mechanical connections to rotate the imaging assembly 120. When used clinically, pullback of the imaging assembly is also automated with a calibrated pullback device (not shown) which operates between coupling 160 and connector 170. Systems for IVUS are also discussed in U.S. Pat. No. 5,771,895; U.S. Pub. 2009/0284332; U.S. Pub. 2009/0195514 A1; U.S. Pub. 2007/0232933; and U.S. Pub. 2005/0249391, the contents of each of which are hereby incorporated by reference in their entirety.

As noted above, the imaging device includes a plurality of markers. In certain aspects of the invention, the plurality of markers is located at a distal region of the device, however, the location can be adjusted as desired. Each marker within the plurality of markers differs from an adjacent marker by size, shape, and/or position on the device.

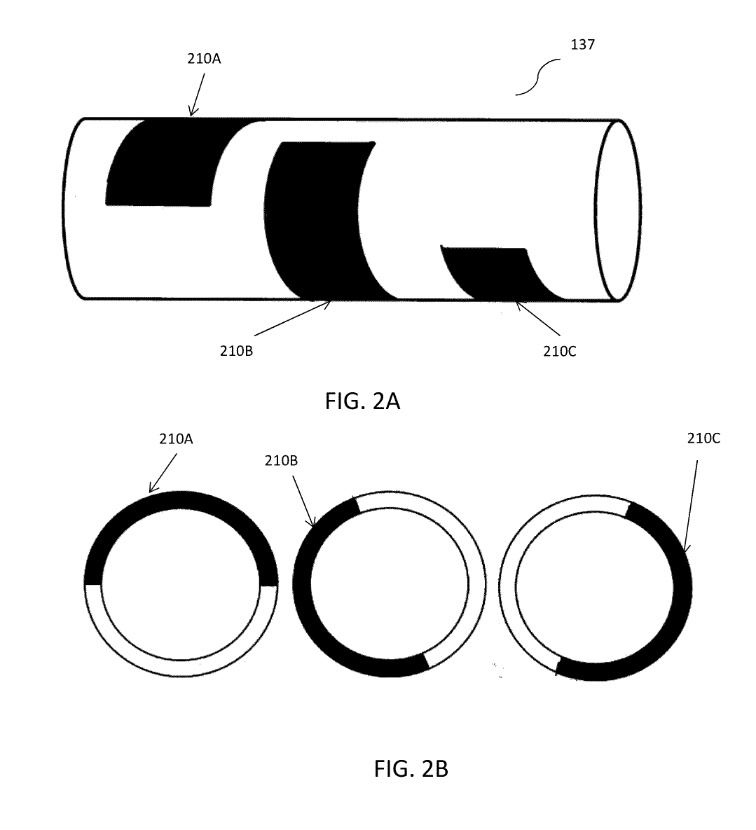

An exemplary embodiment is provided in FIG. 2A, which depicts a close-up of marker region 137 of FIG. 1. As shown, there are three markers, 210A, 210B, and 210C. In this embodiment, each marker is of equal size (half the circumference of the catheter), but differ by their position on the device. More specifically, the markers presented here are offset from one another by an equal amount. In this instance, each marker is offset from the previous by 120 degrees, however, this amount is not limiting. In this embodiment, the difference in the position of each marker will be used to determine the rotational orientation of the device, as explained in further detail below.

FIG. 2B depicts the same three markers from FIG. 2A, but from a forward-facing, cross-sectional view. As shown in FIG. 2B, markers 210A, 210B, and 210C are clearly offset from one another. Although the embodiment depicted in FIGS. 2A and 2B illustrate three markers of equal size, any number of markers may be used. In addition, the shape and size of the markers may differ or may be consistent among the markers. In addition, the markers may be consistently positioned, offset at a consistent degree, or offset by varying degrees. Each of these parameters can be adjusted as desired.

As contemplated by the invention, the markers along the catheter are able to be imaged by an external imaging modality. In certain aspects of the invention, the provided markers are radiopaque markers, which facilitate their imaging by x-ray fluoroscopy or MRI, for instance. In certain aspects of the invention, the radiopaque marker utilizes a radiopaque material, including without limitation, palladium, tungsten, platinum, iridium, borium sulfate, and gold. The nature of the markers can be adjusted as needed depending on the selected imaging modality.

Reference will now be made to an exemplary method using the above device to determine the rotational orientation of the device. Although the method will be explained in further detail below, the method generally comprises providing a device comprising a plurality of markers, wherein each marker within the plurality of markers differs from an adjacent marker by size, shape, and/or position on the device (as exemplified by the device described above. The method further involves imaging the device to capture an image of the device in an imaging plane and processing the captured image to determine an orientation of the device relative to the imaging plane based on said markers. In providing further detail, reference will be made to the device depicted in FIGS. 2A and 2B.

In an exemplary method, the device is an imaging catheter, as shown in FIG. 1. The catheter comprises three markers as shown in FIGS. 2A and 2B. The length of each marker is half the circumference of the catheter and each marker is offset from the other two by 120 degrees. One of the markers (for example, marker 210A of FIGS. 2A and 2B) is selected to be the primary marker and is oriented in a known way when typically using the device. The markers of the imaging catheter are radiopaque, which allows them to be imaged via an external imaging modality, such as fluoroscopy.

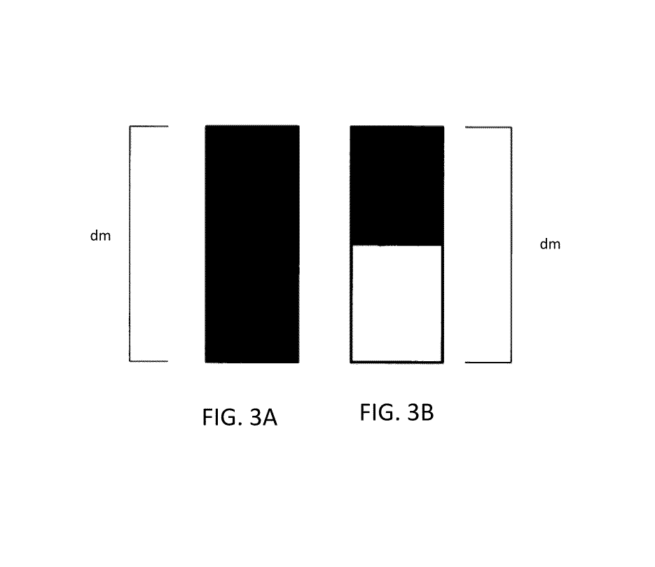

Images from the external imaging system (e.g., fluoroscope) are captured and delivered to the catheter imaging system. By measuring the length of each marker in an image against the known diameter of the catheter, two possible angles of incidence for the imaging plane can be determined. For example, the marker length will only show exactly equal to the catheter diameter dm when the half-circumference of that marker is exactly perpendicular to the direction of imaging, as shown in FIG. 3A. A marker's length will be exactly half the catheter diameter dm when its half circumference is exactly parallel to the direction of imaging, as shown in FIG. 3B. By combining the information from all three markers, a unique orientation of the primary marker can be determined. Based upon this, the orientation of the imaging/treatment system (e.g., IVUS catheter) relative to the direction of the external imaging (e.g., fluoroscopy or angiogram) can be determined. If the orientation of the external imaging plane is known relative to the medial plane of the patient, the orientation of the device can then be calculated in relation to the median plane using, for example, trigonometric methods known in the art.

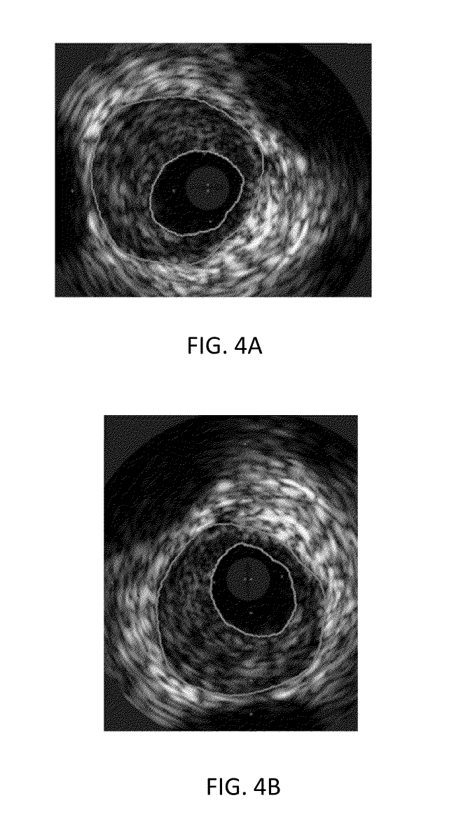

A greatly simplified example of this aspect of the invention is provided in FIGS. 4A and 4B. An IVUS image is obtained from an IVUS catheter, represented by the illustration provided in FIG. 4A. At this stage, it is unknown whether or not the image as shown in in the correct orientation. The image of FIG. 4A was taken by an imaging catheter configured with a plurality of makers as shown in FIGS. 2A and 2B. Marker 210A is selected to be the primary marker and is known to be located on the top of the catheter. When imaging the catheter by fluoroscopy, however, the x-ray image depicts Marker 210A as perpendicular to the imaging plane, as shown in FIG. 3A. This indicates that the catheter was not right-side up at the time the IVUS image was taken, but rather on its side. Examination of Markers 210B and 210C in the external image confirm this conclusion. Accordingly, the rotational orientation of the imaging catheter is known. Subsequently, the orientation of the IVUS image can be appropriately corrected, as shown in FIG. 4B.

In further aspects of the invention, once the orientation of the imaging device is known based on the preceding step, further image processing can be applied to each image captured by the internal imaging device to place it in its proper rotational orientation. This can also be performed using, for example, trigonometric methods known in the art.

It is to be understood that the configuration of markers in the methods just described are not limiting. In other words, other marker configurations are encompassed by the invention. Other embodiments may include for example, a single tight band of markers that extend more than halfway around the catheter diameter but less than 300 degrees. Each marker may be offset by different angles rather than a single consistent angle. This configuration may provide better accuracy when there is significant bending or re-orientation of the device between the two end markers. Other configurations may include a series of markers, where each marker is larger than the preceding marker. Additional configurations encompass markers of different shapes that may be used to distinguish orientation when the device is imaged externally.

Although any catheter, guidewire, and guide catheter can be used in accordance with the invention, in certain embodiments, the catheter is a forward imaging catheter. Extensive detail on forward imaging catheters is provided in U.S. Pat. Nos. 7,736,317; 6,780,157; and 6,457,365, each of which is incorporated by reference herein in its entirety. A catheter-based forward imaging device, whose image is planar, will produce a different image as the catheter is rotated. Nonetheless, it is still important to register and keep track of the imaging plane during cardiovascular procedures. Forward imaging catheters in accordance with the invention solve this problem by using a radiopaque marker with a particular configuration positioned at the distal end. The marker configurations are prepared such that an orientation can be determined by externally viewing the marker. In other words, a part of the configuration would be visible when the device is rotated in one direction relative to an external imaging plane, but not visible when the device is rotated in another direction.

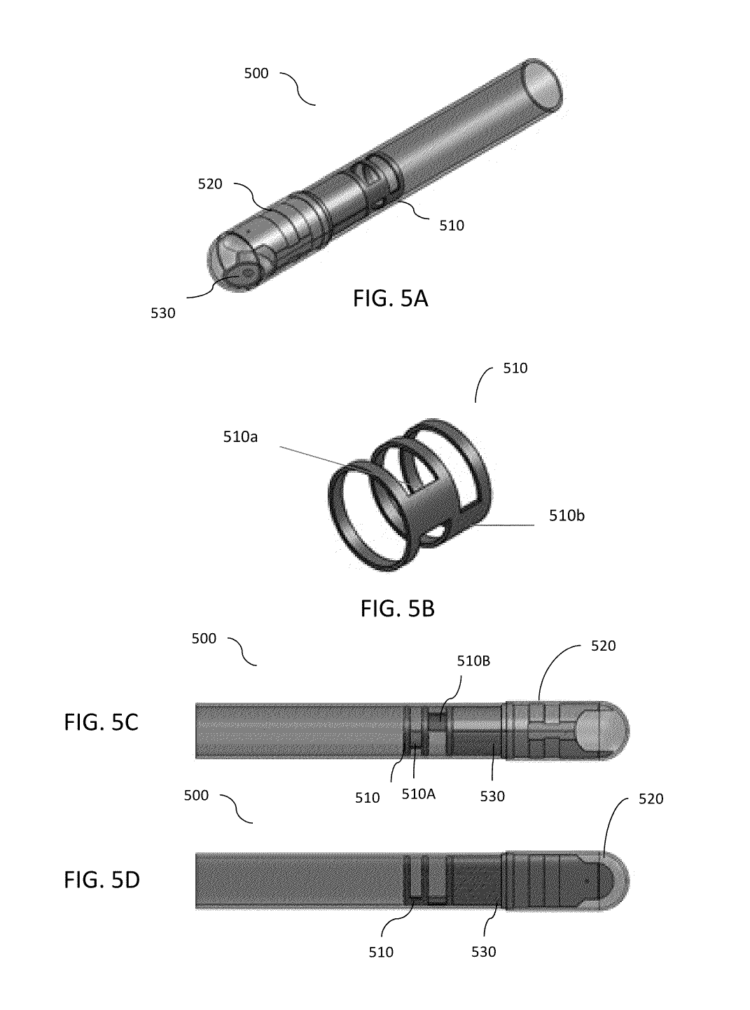

An exemplary forwarding imaging catheter of this kind is depicted in FIGS. 5A-5D. The catheter 500 features a tip 520 at the distal end and an imaging transducer 530 inside the tip 520. The tip 520 may be radiopaque. The imaging transducer 530 can be an ultrasound transducer for IVUS imaging. The imaging transducer 530 can also be optically-based for OCT imaging. The catheter 500 contains a marker component 510 positioned near the distal end of the catheter 500 proximal to the tip 520. The marker component 510 comprises an arrangement of markers whose shape, size, and/or position within the marker component 510 allows the determination of the catheter orientation (as well as any image obtained by the imaging catheter) using the methods descried above. The marker component 510 will appear different when viewed in an external imaging plane (such as an x-ray angiogram), depending on how the catheter 500 is rotationally oriented. For example, when the catheter 500 is positioned right side up, as in FIG. 5C, the external imaging plane depicts two markers 510A and 510B in the marker component 510. When the catheter has been turned on its side (FIG. 5D), however, these two markers 510A and 510B are no longer viewable in the external imaging plane.

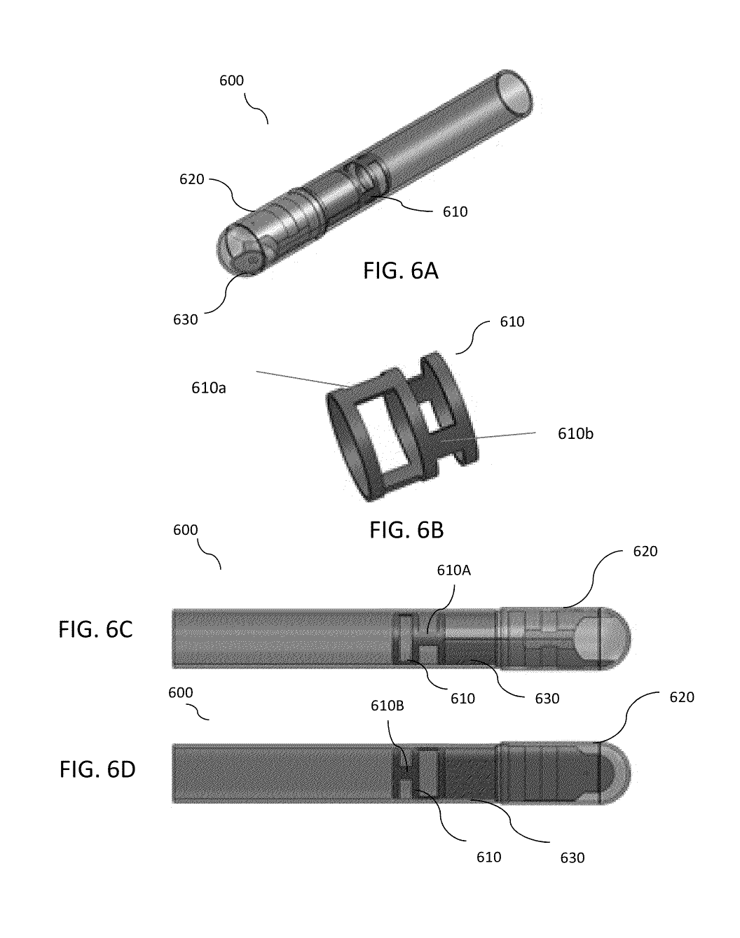

Another exemplary forward imaging catheter is depicted in FIGS. 6A-D. As above, the catheter 600 features a tip 620, an imaging transducer 620, and a marker component 610. The marker component 610 of FIGS. 6A-6D differs from the marker component 510 of FIGS. 5A-5D but still facilitates determination of the rotational orientation, as shown in FIGS. 6C and 6D. As shown, the marker component 620 contains two spatially separated markers 610A and 610B, wherein only one of the two markers is visible when the marker is right-side up (FIG. 6C) or on its side (FIG. 6D).

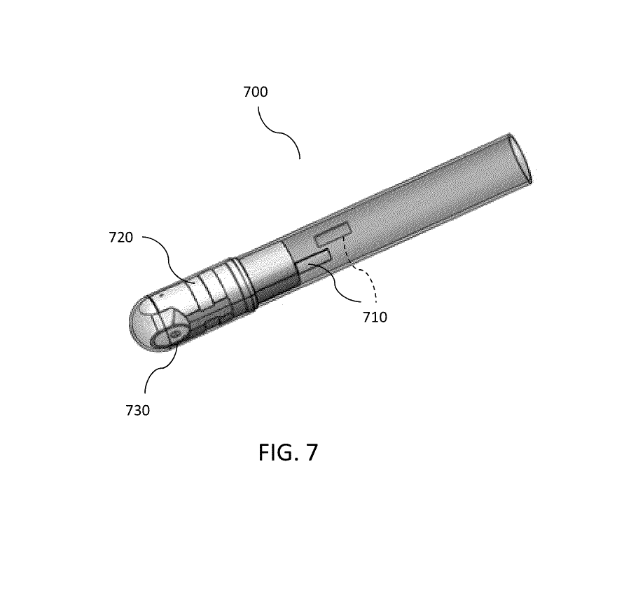

In additional embodiments, the markers are not provided on a separate catheter component, but are etched into the catheter body as shown in FIG. 7. In FIG. 7, the catheter 700 features a tip 720 and an imaging transducer at the distal end. In this embodiment, however, the markers 710 are etched into the body of the catheter 700 rather than provided in a separate component. As shown, the markers 710 are spatially separated and also not on the same plane (in this case, not directly opposite from each other). This spatial separation and offset facilitates determining the orientation of the catheter 700 when viewed externally.

For the catheters depicted in FIGS. 5 and 6, the marker component may be formed by laser cutting a hypotube into the desired configuration, containing an arrangement of markers of a selected number, size, shape, and position. In another aspect, one can use a flat sheet and cut or photo-etch the sheet into the desired configuration and then roll it into its final cylindrical shape. The marker can also be prepared form two different pieces with the individual parts glued together at the distal end of the catheter.

It is contemplated that certain aspects of the invention are particularly amenable for implementation on computer-based systems. Accordingly, the invention also provides systems for practicing the above methods. The system may comprise a processor and a computer readable storage medium instructions that when executed cause the computer to receive a captured image of an externally imaged device comprising a plurality of markers. Each marker within the plurality of markers differs from an adjacent marker by size, shape, and/or position on the device. The instructions also cause the computer to process the captured image to determine an orientation of the device relative to an imaging plane of the captured image. In further embodiments of the provided systems, the instructions additionally cause the computer to determine an orientation of an image captured by the imaging device based on the preceding orientation step.

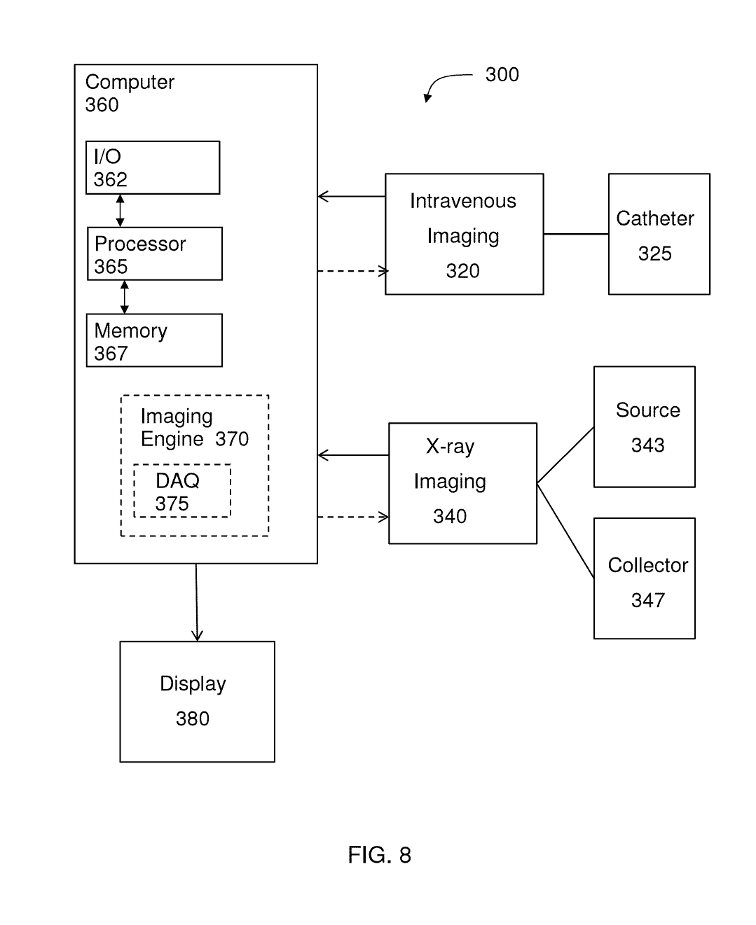

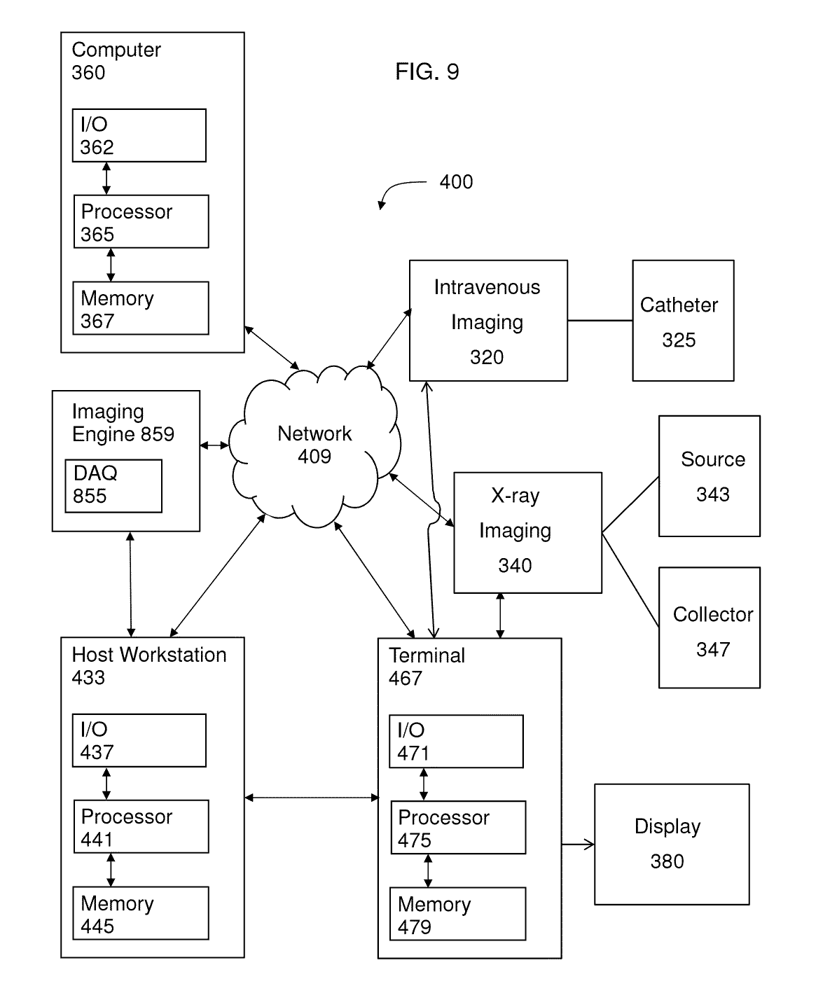

A system of the invention may be implemented in a number of formats. An embodiment of a system 300 of the invention is shown in FIG. 8. The core of the system 300 is a computer 360 or other computational arrangement (see FIG. 9) comprising a processor 365 and memory 367. The memory has instructions which when executed cause the processor to receive imaging data of vasculature of a subject collected with an image collector (e.g., the ultrasonic transducer of an IVUS catheter). The imaging data of vasculature will typically originate from an intravascular imaging device 320, which is in electronic and/or mechanical communication with an imaging catheter 325. The memory additionally has instructions which when executed cause the processor to receive an external image of the catheter including the radiopaque labels. The image of the subject will typically be an x-ray image, such as produced during an angiogram or CT scan. The image of the subject will typically originate in an x-ray imaging device 340, which is in electronic and/or mechanical communication with an x-ray source 343 and an x-ray image collector 347 such as a flat panel detector, discussed above. Having collected the images, the processor then processes the image, and outputs an image of the subject showing the location of the image collector, as well as an image of the vasculature of a subject. The images are typically output to a display 380 to be viewed by a physician or technician. In some embodiments a displayed image will simultaneously include both the intravascular image and the image of the vasculature.

In advanced embodiments, system 300 may comprise an imaging engine 370 which has advanced image processing features, such as image tagging, that allow the system 300 to more efficiently process and display combined intravascular and angiographic images. The imaging engine 370 may automatically highlight or otherwise denote areas of interest in the vasculature. The imaging engine 370 may also produce 3D renderings of the intravascular images and or angiographic images. In some embodiments, the imaging engine 370 may additionally include data acquisition functionalities (DAQ) 375, which allow the imaging engine 370 to receive the imaging data directly from the catheter 325 or collector 347 to be processed into images for display.

Other advanced embodiments use the I/O functionalities 362 of computer 360 to control the intravascular imaging 320 or the x-ray imaging 340. In these embodiments, computer 360 may cause the imaging assembly of catheter 325 to travel to a specific location, e.g., if the catheter 325 is a pull-back type. The computer 360 may also cause source 343 to irradiate the field to obtain a refreshed image of the vasculature, or to clear collector 347 of the most recent image. While not shown here, it is also possible that computer 360 may control a manipulator, e.g., a robotic manipulator, connected to catheter 325 to improve the placement of the catheter 325.

A system 400 of the invention may also be implemented across a number of independent platforms which communicate via a network 409, as shown in FIG. 6. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

As shown in FIG. 9, the intravascular imaging system 320 and the x-ray imaging system 340 are key for obtaining the data, however the actual implementation of the steps, for example the steps of FIG. 6, can be performed by multiple processors working in communication via the network 409, for example a local area network, a wireless network, or the internet. The components of system 400 may also be physically separated. For example, terminal 467 and display 380 may not be geographically located with the intravascular imaging system 320 and the x-ray imaging system 340.

As shown in FIG. 9, imaging engine 859 communicates with host workstation 433 as well as optionally server 413 over network 409. In some embodiments, an operator uses host workstation 433, computer 449, or terminal 467 to control system 400 or to receive images. An image may be displayed using an I/O 454, 437, or 471, which may include a monitor. Any I/O may include a monitor, keyboard, mouse, or touch screen to communicate with any of processor 421, 459, 441, or 475, for example, to cause data to be stored in any tangible, nontransitory memory 463, 445, 479, or 429. Server 413 generally includes an interface module 425 to communicate over network 409 or write data to data file 417. Input from a user is received by a processor in an electronic device such as, for example, host workstation 433, server 413, or computer 449. In certain embodiments, host workstation 433 and imaging engine 855 are included in a bedside console unit to operate system 400.

In some embodiments, the system may render three dimensional imaging of the vasculature or the intravascular images. An electronic apparatus within the system (e.g., PC, dedicated hardware, or firmware) such as the host workstation 433 stores the three dimensional image in a tangible, non-transitory memory and renders an image of the 3D tissues on the display 380. In some embodiments, the 3D images will be coded for faster viewing. In certain embodiments, systems of the invention render a GUI with elements or controls to allow an operator to interact with three dimensional data set as a three dimensional view. For example, an operator may cause a video affect to be viewed in, for example, a tomographic view, creating a visual effect of travelling through a lumen of vessel (i.e., a dynamic progress view). In other embodiments an operator may select points from within one of the images or the three dimensional data set by choosing start and stop points while a dynamic progress view is displayed in display. In other embodiments, a user may cause an imaging catheter to be relocated to a new position in the body by interacting with the image.

In some embodiments, a user interacts with a visual interface and puts in parameters or makes a selection. Input from a user (e.g., parameters or a selection) are received by a processor in an electronic device such as, for example, host workstation 433, server 413, or computer 449. The selection can be rendered into a visible display. In some embodiments, an operator uses host workstation 433, computer 449, or terminal 467 to control system 400 or to receive images. An image may be displayed using an I/O 454, 437, or 471, which may include a monitor. Any I/O may include a keyboard, mouse or touch screen to communicate with any of processor 421, 459, 441, or 475, for example, to cause data to be stored in any tangible, nontransitory memory 463, 445, 479, or 429. Server 413 generally includes an interface module 425 to effectuate communication over network 409 or write data to data file 417. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 433 and imaging engine 855 are included in a bedside console unit to operate system 400.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell networks (3G, 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Per1), and it can be deployed in any form, including as a stand-alone program or as a module, component, subroutine, or other unit suitable for use in a computing environment. Systems and methods of the invention can include programming language known in the art, including, without limitation, C, C++, Per1, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties so that optical read/write devices can then read the new and useful collocation of information (e.g., burning a CD-ROM). In some embodiments, writing a file includes using flash memory such as NAND flash memory and storing information in an array of memory cells include floating-gate transistors. Methods of writing a file are well-known in the art and, for example, can be invoked automatically by a program or by a save command from software or a write command from a programming language.

In certain embodiments, display 380 is rendered within a computer operating system environment, such as Windows, Mac OS, or Linux or within a display or GUI of a specialized system. Display 380 can include any standard controls associated with a display (e.g., within a windowing environment) including minimize and close buttons, scroll bars, menus, and window resizing controls. Elements of display 380 can be provided by an operating system, windows environment, application programming interface (API), web browser, program, or combination thereof (for example, in some embodiments a computer includes an operating system in which an independent program such as a web browser runs and the independent program supplies one or more of an API to render elements of a GUI). Display 380 can further include any controls or information related to viewing images (e.g., zoom, color controls, brightness/contrast) or handling files comprising three-dimensional image data (e.g., open, save, close, select, cut, delete, etc.). Further, display 380 can include controls (e.g., buttons, sliders, tabs, switches) related to operating a three dimensional image capture system (e.g., go, stop, pause, power up, power down).

In certain embodiments, display 380 includes controls related to three dimensional imaging systems that are operable with different imaging modalities. For example, display 380 may include start, stop, zoom, save, etc., buttons, and be rendered by a computer program that interoperates with IVUS, OCT, or angiogram modalities. Thus display 380 can display an image derived from a three-dimensional data set with or without regard to the imaging mode of the system.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. An intraluminal device comprising a plurality of detectable markers, wherein each marker within the plurality of markers differs from an adjacent marker by size, shape, and/or position on the device.

2. The device of claim 1, wherein the position of at least one of said markers is rotationally offset with respect to an adjacent marker.

3. The device of claim 1, wherein the plurality of markers is located on a component attached to the device.

4. The device of claim 1, wherein said plurality comprising at least three markers.

5. The device of claim 1, wherein the device is an imaging device.

6. The device of claim 1, wherein the imaging device is an intravascular ultrasound imaging device.

7. The device of claim 1, wherein the imaging device is an optical coherence tomography imaging device.

8. The device of claim 1, wherein the device is a catheter.

9. The device of claim 1, wherein the catheter is a forward imaging catheter.

10. The device of claim 1, wherein the device is a guidewire.

11. The device of claim 1, wherein the marker is a radiopaque marker.

12. The device of claim 1, wherein the radiopaque marker is selected from a group consisting of palladium, tungsten, platinum, iridium, borium sulfate, and gold.

13. A method for determining the orientation of a device, the method comprising:
providing a device comprising a plurality of markers, wherein each marker within the plurality of markers differs from an adjacent marker by size, shape, and/or position on the device;
inserting the device into a vessel;
imaging the device to capture an image of the device in an imaging plane; and
processing the captured image to determine an orientation of the device relative to the imaging plane based on said markers.

14. The method of claim 13, wherein said orientation is a rotational orientation.

15. The method of claim 13, wherein the device is an imaging device.

16. The method of claim 15, wherein the imaging device is an intravascular ultrasound imaging device.

17. The method of claim 15, wherein the imaging device is an optical coherence tomography imaging device.

18. The method of claim 13, wherein the device is a catheter.

19. The method of claim 13, wherein the catheter is a forward imaging catheter.

20. The method of claim 13, wherein the plurality of markers is located on a component attached to the device.

21. The method of claim 13, wherein the device is a guidewire.

22. The method of claim 15, further comprising orienting an image captured by said imaging device based on the prior orientation step.

23. The method of claim 13, wherein the device comprises at least three markers within the plurality of markers.

24. The method of claim 15, wherein imaging the device comprises fluoroscopic imaging of the device.

25. A system for determining the orientation of a device, comprising:
a processor; and
a computer readable storage medium instructions that when executed cause the computer to:
receive a captured image of an externally imaged device comprising a plurality of markers, wherein each marker within the plurality of markers differs from an adjacent marker by size, shape, and/or position on the device; and
process said captured image to determine an orientation of the device relative to an imaging plane of the captured image.

26. The system of claim 25, wherein said orientation comprises a rotational orientation.

27. The system of claim 25, wherein said device is an imaging device.

28. The system of claim 27, further comprising determining an orientation of an image captured by said imaging device based on the prior orientation step.

29. The system of claim 27, wherein the imaging device comprises an intravascular ultrasound imaging device.

30. The system of claim 27, wherein the imaging device comprises an optical coherence tomography imaging device.
